(19) United States
(12) Patent Application Publication
(10) Pub. No.: US 2025/0073051 A1
(43) Pub. Date: Mar. 6, 2025

(54) Change-speed gearbox for orthopedic devices

(71) Applicant: Otto Bock Healthcare Products GmbH, Wien (AT)

(72) Inventor: Thomas West PICKERILL, Wien (AT)

(21) Appl. No.: 18/806,960

(22) Filed: Aug. 16, 2024

(30) Foreign Application Priority Data
Aug. 28, 2023 (DE) 10 2023 123 055.7

(51) Int. Cl. A61F 2/68; A61F 2/50

(52) U.S. Cl. CPC A61F 2/68; A61F 2002/5007; A61F 2002/6836

(57) ABSTRACT

A change-speed gearbox for orthopedic devices has a planetary gearbox with at least one planet stage having a central sun gear, a plurality of planet gears rotatably mounted on a planet carrier which come into engagement with the sun gear, and has a ring gear which meshes with the planet gears. The sun gear can be coupled to a drive. The planet carrier is coupled to an output element. The ring gear is pivotably mounted in a ring gear carrier and is coupled to the ring gear carrier via a switchable freewheel.

### FIELD OF THE INVENTION

The invention relates to a change-speed gearbox for orthopedic devices, for example orthoses, prostheses or other orthopedic devices, having a planetary gearbox with at least one planet stage having a central sun gear, having a plurality of planet gears rotatably mounted on a planet carrier, which come into engagement with the sun gear, and having a ring gear which meshes with the planet gears, wherein the sun gear can be coupled to a drive and the planet carrier is coupled to an output element.

### BACKGROUND

Orthopedic devices, in particular orthoses, exoskeletons and prostheses, frequently have components which are mounted on one another so as to be pivotable or displaceable. To influence the relative movement between the components, for example an upper part and a lower part of an orthosis spanning a joint or a prosthesis having a joint, different measures are taken. Mechanical stops can be provided to limit the maximum pivoting angle or displacement travel. In order to influence the pivoting movement or displacement movement, energy stores or dampers can be arranged between the two components as passive influencing devices.

Active orthopedic devices have a drive which initiates a relative movement between the components, assists it or opposes a resistance to the relative movement, in order to brake or to prevent the relative movement. Electric motors are often provided for this purpose, which are activated, deactivated or modulated by a control device. The electric motor is coupled to an accumulator or a battery in order to permit appropriate active influencing or initiation of a relative movement.

Both for the energy store and for the drive in the form of motors, the space that is available in orthopedic devices is limited. Motors which can generate large moments at high rotational speeds are often too heavy, in particular in orthopedic devices of the upper extremity. Therefore, as a rule small, high-speed motors are used, which are coupled via a gearbox to the component respectively to be driven or to be influenced.

In order to be able to apply both a fast displacement and also a high torque with a comparatively small drive, a change-speed gearbox which is able to switch between a higher and a lower gear is advantageous. In a higher gear, the gearbox permits higher rotational speeds with comparatively lower torques. Such a switching position is advantageous, for example, for the swing phase of an orthopedic device of the lower extremity. In a low gear, higher extension torques with lower displacement speeds are possible which, for example in an orthopedic device of the lower extremity, is advantageous for the stance phase, for standing up or when climbing stairs.

US 2021/0338458 A1 discloses an orthopedic device in the form of an exoskeleton or a prosthetic knee joint, in which an actuating mechanism is coupled with an artificial knee joint in order to pivot the artificial knee joint. A motor drives the actuating mechanism, a transmission mechanism with a variable crank can provide a first torque and speed profile for the pivoting movement. In a second torque and speed profile, which is different from the first profile, a greater torque is provided at lower speeds. The crank has, for example, an adjustable length.

EP 4 166 287 A1 describes, inter alia, an active prosthetic device having a joint device with two parts and a connecting unit, which connects the two parts pivotably to one another. An expansion device and a contraction device are configured to change the angle between the two parts. The expansion and contraction device has an energy source and a transmission unit for transmitting the power. The transmission unit has two transmission paths with different transmission ratios. Switching cams slide along a plunger, which can lead to comparatively high wear and potential jamming situations.

The dissertation “A Primarily Passive Knee Prosthesis with Powered Stance and Swing Assistance”, Stephen Christopher Culver, Vanderbilt University, Nashville, Tennessee, Dec. 17, 2022, discloses a prosthetic knee joint having swing phase support and a driven stance phase, in which a two-stage gearbox is used in order once to be able to transmit high rotational speeds with a low torque and once to transmit a high torque at low rotational speeds. For this purpose, a planetary gearbox having two controllable clutches is provided. Each clutch has a separate mechanism, which is controlled via a microprocessor.

### SUMMARY

It is an object of the present invention to provide a change-speed gearbox which, with little noise and low losses, can be used flexibly and reliably and permits a rapid changeover.

The object is achieved by a change-speed gearbox for orthopedic devices having the features of the main claim. Advantageous refinements and developments are disclosed in the subordinate claims, the description and the figures.

The change-speed gearbox for orthopedic devices, having a planetary gearbox with at least one planet stage having a central sun gear, having a plurality of planet gears rotatably mounted on a planet carrier, which come into engagement with the sun gear, and having a ring gear which meshes with the planet gears, wherein the sun gear can be coupled to a drive and the planet carrier is coupled to an output element, is characterized in that the ring gear is pivotably mounted in a ring gear carrier and is coupled to the ring gear carrier via a switchable freewheel. The ring gear is mounted in the ring gear carrier, in particular such that it can pivot or rotate about the axis of rotation of the sun gear or about an axis perpendicular to the plane in which the planet gears rotate. As a result, the ring gear can be rotated overall. The change-speed gearbox therefore permits at least two different transmission ratios for the drive in the orthopedic device and the transmission of torques from the drive to the components to be driven. The change-speed gearbox changes between a high gear with high rotational speeds and low torques and a low gear with low rotational speeds and higher torques. By means of the switchable freewheel, it is possible firstly to change over between the high gear and the low gear and, secondly, during a reverse movement from the output element to the change-speed gearbox, to perform the transmission of the rotational speed to the drive always in the chosen gear, in particular in a high gear, as a result of which, for example, the drive can effectively be operated in the form of an electric motor in generator operation.

In one embodiment, the freewheel can be torque-controlled, so that a change between a high gear and a low gear can be carried out without an electronic control device and without sensors when an adjustable or set torque is exceeded. This torque is the drive torque which is applied by the drive and with which the drive is assisted. The load-dependent, automatic changeover saves installation space, components and weight. Alternatively, the freewheel is changed over between the different positions via an actuator.

In a development, a controllable clutch is arranged between the sun gear and the output element, via which it is possible to uncouple and to connect the drive from and to the output element. As a result, for example when, with an unlocked freewheel, the ring gear can be rotated freely relative to the ring gear carrier, it is possible for the gearbox to be uncoupled from the drive and for no torque to be transmitted between the drive and the output element.

In one embodiment, the output element is axially displaceably mounted on the planet carrier, so that the output element can be moved in the direction of the sun gear and away from the same. Between the sun gear and the output element, in an engaged position, in one embodiment the controllable clutch arranged between the same transmits the torque. The controllable clutch is, for example, designed as a slipping clutch, claw clutch, cone clutch or the like and couples or uncouples the output element from the drive.

In one embodiment, the output element is spring-biased in the direction of the sun gear, so that as a basic setting and without the application of further forces, the controllable clutch is engaged and the components of the clutch are urged into an engaged position.

In one embodiment, the output element is mounted on a thread or torsionally rigidly and axially displaceably on an output component, for example a driveshaft. By means of the coupling via a thread, in particular in combination with a spring bias of the output element on the output component, it is possible to permit torque-dependent activation of the controllable clutch, for example the controllable slipping clutch, claw clutch or cone clutch.

In one embodiment, the output element is coupled to a sleeve that is rotatable relative to the ring gear carrier, via which it is possible to move the freewheel into a force-transmitting engaged position or into a released position. In one embodiment, the control sleeve is coupled to the output element and, by means of a rotation relative to the ring gear carrier in conjunction with a thread, can achieve an axial displacement and/or rotation of the output element. In the case of an axially displaceable, torsionally rigid mounting of the output element on the output component, the control sleeve is advantageously arranged on a thread on the ring gear carrier or a component arranged or designed to be fixed relative thereto, so that a rotation simultaneously means an axial displacement. By means of a rotational mounting of the control sleeve on the output element, a linear movement of the output element away from the sun gear or toward the sun gear is then effected, possibly assisted by a spring. When the control element is mounted on a thread on the output component, the rotation of the control sleeve can cause an axial displacement in one or the other direction via the thread, depending on the direction of rotation.

In one embodiment, the freewheel has at least one blocking element, which is spring-loaded in the direction of an engaged position, in which the blocking element comes into force-transmitting engagement with the ring gear and the ring gear carrier. The blocking element is assigned at least one control pin, which moves the blocking element counter to the spring force into a released position, in which the blocking element is out of engagement. In particular, a plurality of blocking elements and a plurality of control pins are assigned to one another, in particular each blocking element is assigned a control pin in order to move the blocking element into the respective desired position. The embodiment of the blocking element or of the blocking elements can depend on the design of the freewheel; in particular the blocking elements are clamping elements or pawls, alternatively the blocking element is a wrap spring, which is configured to be controllable.

In one embodiment, the control pin or the control pins are mounted on the control sleeve that is rotatable relative to the ring gear carrier or a stator carrier that is rotatable relative to the ring gear carrier. The stator carrier is part of the drive and, in particular, is rotatably mounted on a housing or a component that is coupled in a fixed manner to the ring gear carrier. The arrangement of the control pins on the moving stator carrier makes it possible that, even without an actuator or a motor, the freewheel and therefore the planet stage can be configured to be torque-dependent. The control pins or the control pin displace the blocking element or the blocking elements as a function of the torque applied by the drive and the necessary support of the stator on the housing or a component that is coupled in a fixed manner to the ring gear carrier. An automatic and torque-dependent changeover between a high gear with a high rotational speed and low torque and a low gear with a low rotational speed and a high torque, if appropriate in conjunction with a clutch that can be controlled as a function of torque, permit a compact design. The output element or the output components can permit at least two different transmission ratios in one output direction of rotation, while in the other direction of rotation there is always a standard transmission ratio, in particular a direct transmission in which no change in rotational speed takes place. The standard transmission ratio can also include a rotational speed change.

In another embodiment, the control sleeve is coupled to the actuator, so that controlled, in particular electronically controlled, activation, modulation or deactivation of the actuator and therefore a displacement of the control sleeve can be made on the basis of sensor data. Via the actuator, different positions of the control sleeve can be reached; in particular the clutch can be disengaged or engaged via the actuator and the control pin or the control pins can be brought into engagement or out of engagement with the blocking element or the blocking elements in order to effect a changeover between an engaged position and a release position.

The control sleeve can be coupled to the actuator by a frictional fit or via toothing, toothing permits a very precise adjustment, while a frictional transmission of force of the actuator with the control sleeve can protect the components against mechanical overload.

In one embodiment, the control sleeve has an adjustment thread which meshes with a carrier thread so that, during a rotation of the control sleeve, at the same time an axial displacement takes place, so that in particular the output element can be displaced relative to the planetary gearbox and to the output components.

In one embodiment, the carrier thread is arranged or formed on the ring gear carrier or on a component coupled in a fixed manner to the ring gear carrier.

In one embodiment, the control sleeve is formed in such a way that, in a first position of the control sleeve, the clutch is engaged and the freewheel is kept out of engagement. The controllable clutch is engaged in a force-transmitting manner, the planetary gearbox has no influence on the transmission ratio because of the freewheel in the released position, so that a direct transmission of force and a transmission of rotational speed from the drive to the output element and therefore to the output components, in particular a driveshaft, is carried out.

In one embodiment, the change-speed gearbox is designed such that, in a second position of the control sleeve, the clutch is disengaged and the freewheel is additionally kept out of engagement. In this position, an intermediate position between the two gears, the output is completely uncoupled from the drive. Were the change-speed gearbox to be arranged between two joint components, complete uncoupling of the change-speed gearbox from the two pivotable components would be carried out. There would be no influence on the movement by the drive and the change-speed gearbox.

In a development, in a third position of the control sleeve, the clutch is disengaged and the freewheel is kept in engagement. As a result, a low gear is provided by the planetary gearbox. At a low rotational speed, a high torque is transmitted from the drive to the output element or the output components.

In one embodiment, the stator carrier is biased in the direction of an initial position via an energy store, e.g. a spring, wherein in the initial position the freewheel is kept out of engagement, in particular the clamping elements or blocking elements are brought out of engagement, so that a low torque to be applied by the drive, for example in the swing phase, ensures the free mobility of the ring gear, as a result of which there is direct transmission from the drive to the output element. The freewheel is only blocked in the event of a high resistance, for example in the stance phase, therefore the ring gear is blocked and the slipping clutch is automatically disengaged as the force rises, so that the transmission of the forces from the drive takes place via the planet stage. Given such a mechanical embodiment, there can be a transmission range in which the system is blocked very briefly if the slipping clutch is not yet disengaged. Only as the torque rises does the clutch begin to slip and the spring is tensioned, so that there is no longer any contact.

In one embodiment, the stator carrier is mounted on the ring gear carrier or on a component coupled in a fixed manner to the ring gear carrier.

In one embodiment, the planet stage is designed as a toothed gear stage, in which the sun gear has external toothing and the planet gears are provided with external toothing systems which mesh with the external toothing of the sun gear, in which the ring gear has internal toothing which meshes with the external toothing of the planet gears.

In a further embodiment, the change-speed gearbox can be changed over between two transmission ratios in a first direction of rotation and, in the opposite, second direction of rotation, is switched to a non-switchable standard transmission ratio, so that a drive can be driven both quickly and also slowly and with a greater torque in a first adjustment direction, for example, of an orthosis or prosthesis. In the other adjustment direction, there is another direction of rotation of the change-speed gearbox because of the reversed direction of rotation of the drive which takes place with the standard transmission ratio, which is adapted and optimized to the conditions usually present for this adjustment direction. For example, an extension movement can be provided with a two-stage change-speed gearbox which changes over between a high and a low gear, while for the flexion movement, a high transmission ratio is always provided for rapid adjustment. The standard transmission ratio can be a direct transmission or effect a change in rotational speed from the drive to the output components.

### DESCRIPTION OF THE DRAWINGS

Exemplary embodiments of the invention are explained in more detail below by using the figures, in which:

FIG. 1 shows a schematic illustration of a first variant of the change-speed gearbox with a motor actuator;

FIG. 2 shows various switching positions of the gearbox;

FIG. 3 shows an illustration of an uncoupled freewheel;

FIG. 4 shows an illustration of the coupled change-speed gearbox;

FIG. 5 shows an illustration with freewheel in the engaged position; and

FIG. 6 shows a variant with an elastically supported stator.

### DETAILED DESCRIPTION

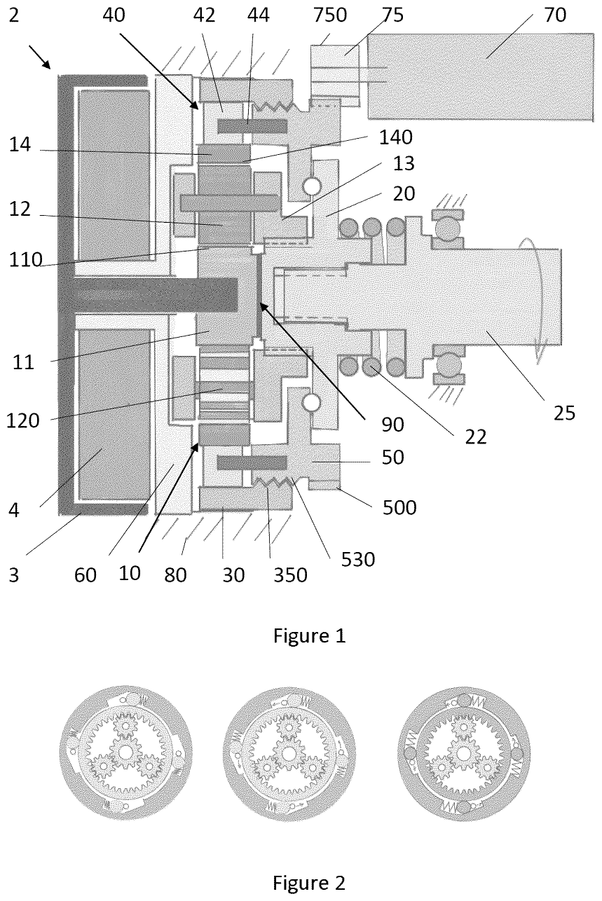

A change-speed gearbox for an orthopedic device is illustrated in a schematic sectional illustration in FIG. 1. The orthopedic device is, for example, an orthosis or prosthesis, in particular a prosthesis of the lower extremity having a joint device which fixes two components of the orthopedic device pivotably to one another. A drive 2 having a rotor 3 under a stator 4 is arranged via a stator carrier 60 on a stationary component 80, for example an upper part of an orthopedic joint device. The drive 2, which is designed as an electric motor, is coupled via a planetary gearbox 10 to an output component 25, which is set rotating by the drive 2 and drives the lower part fixed in an articulated manner to the upper part, either directly or via a force transmission device. As an alternative to an embodiment as a drive for an orthosis or prosthesis of the lower extremity, the drive 2 having the change-speed gearbox can be used as a drive for a prosthetic hand, for example.

The rotor 3 of the drive 2 is coupled via a drive pin to a sun gear 11 of the planetary gearbox 10. The planetary gearbox 10 is constructed conventionally and, in addition to the central sun gear 11, has a plurality of planet gears 12 arranged around the same, which are rotatably mounted on a planet carrier 13. The planet gears 12 revolve in a ring gear 14 which, in the exemplary embodiment illustrated, is coupled to a ring gear carrier 30 via a switchable freewheel 40. The freewheel 40 has a plurality of blocking elements 42 distributed around the circumference of the ring gear 14, each of which is coupled to a control pin 44. In the exemplary embodiment illustrated, the blocking element 42 is designed as a cylindrical or spherical clamping element, which is arranged on the outside of the ring gear 14 and inside a recess in the ring gear carrier 30. Arranged inside the recess is a spring, which cannot be seen in FIG. 1. The spring urges the blocking element 42 into a blocking position, in which the ring gear 14 is blocked with respect to the ring gear carrier 30. The ring gear carrier 30 itself is fixed in the stationary component 18, for example the upper part. Alternatively, the freewheel 40 is formed directly between the ring gear 14 and the stationary component 80. The interposition of a ring gear carrier 30 permits simplified fabrication of the internal geometry of the ring gear carrier 30 and makes the selection of suitable materials easier.

Via the control pins 44, it is possible and intended to move the respective blocking elements 42 counter to a spring force into a released position, in which the ring gear 14 can rotate freely. The adjustment or switching of the control pins 44 is carried out via a control sleeve 50, in which the control pins 44 are likewise arranged. The control sleeve 50 is equipped on the outside with an adjustment thread 530, which meshes with a corresponding carrier thread 350 in the ring gear carrier 30. An actuator 70 having a gear wheel 75 and external toothing 750 engages in toothing 500 of the control sleeve likewise arranged on the outside. If the actuator 70, for example an electric motor, is activated, the gear wheel 75 rotates in one or the other direction. Accordingly, because of the two sets of toothings 750, 500, the controlled sleeve rotates in the paired threads comprising the carrier thread 350 and the adjusting thread 530. As a result of a rotation, the result is firstly an adjustment of the control pins 44 in the circumferential direction and, at the same time, an axial displacement of the control sleeve 50 because of the thread pitch.

The control sleeve 50 is additionally mounted via a spherical bearing on an output element 20 which can be coupled in a force-transmitting manner by a front face facing the sun gear 11 to the sun gear 11 via a controllable clutch 90. Furthermore, the output element 20 has an extension, on which toothing, for example a spline, is formed. The spline meshes with corresponding internal toothing on the planet carrier 13, so that there is an axially displaceable, torsionally rigid coupling between the planet carrier 13 and the output element 20. The output element 20 additionally has a bore and a flange. Toothing, for example polygonal toothing or a spline, which meshes with corresponding external toothing on a pin of the output component 25, is likewise formed in the central bore. The output component 25 is rotatably mounted, in particular axially supported, in a bearing on the stationary component 80. Likewise formed on the output component 25 is an extension, on which a compression spring 22 is supported and which is supported by its other end on the flange of the output element 20. The output element 20 is thus torsionally rigidly and axially displaceably mounted on the output component 25 and biased in the direction of the planetary gearbox 10 via the compression spring 22.

In the exemplary embodiment illustrated, the planetary gearbox 10 is formed as a toothed gearbox and has a single planet stage, in which the sun gear 11 has external toothing 110, the planet gears 12 have external toothing 120, and the ring gear 14 has internal toothing 140. The sets of external toothings 110, 120 mesh with one another, the external toothings 120 of the planet gears 12 mesh with the internal toothing 140 of the ring gear 14. As an alternative to an embodiment as a toothed gearbox, the planetary gearbox 10 is designed as a friction gearbox.

The change-speed gearbox according to FIG. 1 makes it possible to set a plurality of switching positions with only one actuator 70. In addition to direct coupling of the output element 20 via the controllable clutch 90 to the rotor 3 of the drive 2, which results in a high rotational speed with a low torque on the output component 25, there is the possibility of blocking the ring gear 14 within the ring gear carrier 30. The force is then transmitted via the planet carrier 13 to the external toothings of the output element 20, by which means an increased torque is provided at a reduced rotational speed. In an intermediate position, the drive 2 is uncoupled completely from the output element 20, in that the controllable clutch 90 is disengaged and the blocking elements 40 are set into a released position with respect to the control pins 44.

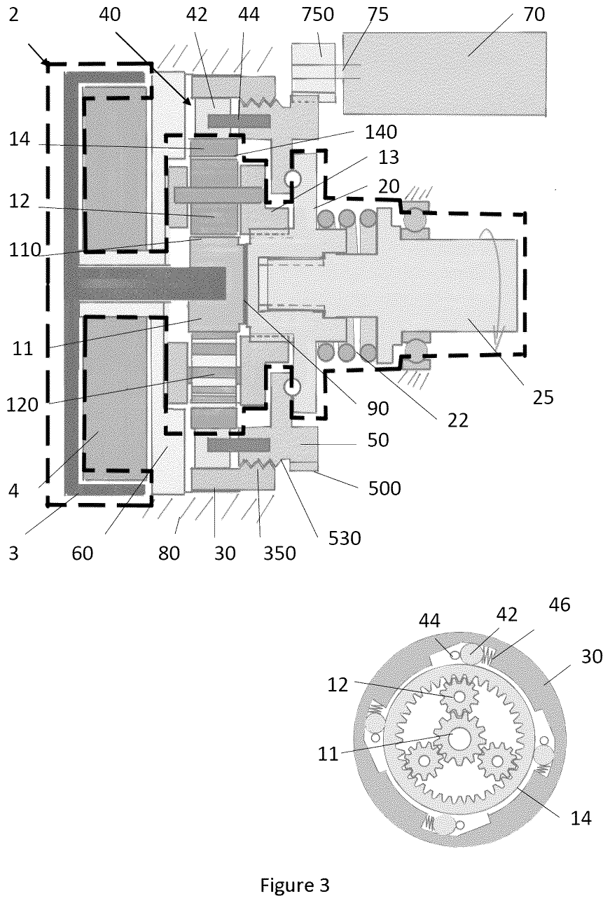

In FIG. 2, the three positions described above are shown in a cross-sectional view through the planetary gearbox. In the figures, the ring gear carrier 30 having the recesses for the blocking elements 42 in the form of balls or rollers supported on springs 46 can be seen. The blocking elements 42 are each assigned a control pin 44, which are fastened in the control sleeve 50, not illustrated, and can be rotated about the common axis of rotation of the motor 2, of the sun gear 11, of the output element 20 and of the output component 25. In the left-hand illustration of FIG. 2, the situation is substantially illustrated in which the control sleeve 50 has been screwed axially as far as possible into the ring gear carrier 30, wherein the adjusting thread 530 and the carrier thread 350 are depicted as right-hand threads. In the position, the inwardly projecting extension of the control sleeve 50 is located closest to the planetary gearbox 10. Via the spring 22, the output element 20 on the toothing of the pin of the output component 25 is urged in the direction of the sun gear 11. The coupling 90 is engaged, the springs 46 are compressed to the maximum and the blocking elements 42 are retained in the ring gear carrier 30 via the control pins 44 inside the radially widening pockets. A force is transmitted, as shown in FIG. 3, from the rotor 2 via the sun gear 11 and the clutch 90 directly to the output element 20 and via the form-fitting, axially displaceable coupling of the toothing to the output component 25. The ring gear 40 is freely rotatably supported within the ring gear carrier 30.

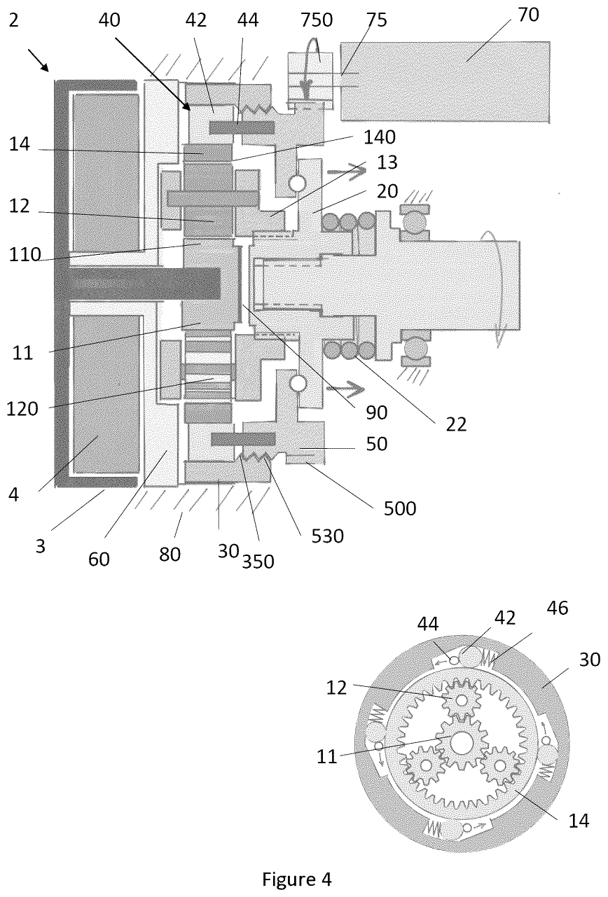

In the central position of FIG. 2, which is illustrated in FIG. 4, the actuator 70 has been activated, the gear wheel 75 has been driven and the toothing 750, which is engagement with the toothing 500 of the control sleeve 50, has been screwed out of the ring gear carrier 30. Because of the rotation of the control sleeve 50 and an adjustment of the control pins 42 in the circumferential direction, which is indicated in FIG. 4 by the arrow in the lower illustration, an axial displacement of the output element 20 away from the planetary gearbox 10 takes place. This is indicated by the arrows in the upper illustration of FIG. 4. The controllable clutch 90 is disengaged, the output element 20 is separated from the sun gear 11 and no transmission of force takes place, since the driven sun gear 11 does not drive the output element 20 via the clutch 90. The external toothing 110 of the sun gear 11 drives the external toothings 120 of the planet gears 12, which in turn rotate the ring gear 14 which, because of the position of the control pins 44 and the non-blocking locking elements 42, is freely rotatable within the ring gear carrier 30. The planet carrier 13 is held in position via the resistance torque, which counteracts an adjustment by the output component.

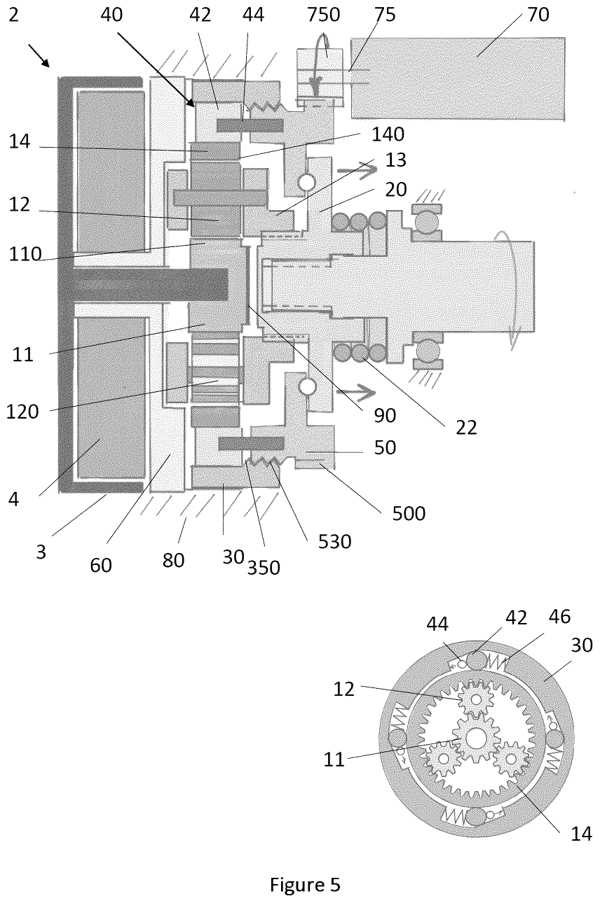

The right-hand position in FIG. 2 of the switchable freewheel 40 is shown in FIG. 5. The actuator 70 has been activated further, the control sleeve 50 has been rotated further and the control pins 44 have rotated further to the left in the counter-clockwise direction, as shown in the lower illustration in FIG. 5. As a result, blocking elements 42 are pressed via the spring 46 and possibly also via friction into the chambers of the ring gear carrier 30 that taper radially in the circumferential direction. As a result, the ring gear 14 and the ring gear carrier 30 have wedged or jammed, as a result of which the planet gears 12 revolve within the internal toothing 140 of the ring gear 14 and drive the planet carrier 13. Because of the transmission ratio, the planet carrier 13 is driven with a lower rotational speed than the rotational speed of the drive 2 but with a high torque, so that a changeover from a high gear of the position according to FIG. 2 to a low gear of the position according to FIG. 5 results. In the intermediate position according to FIG. 4, there is no transmission of torque or of force.

In principle, the change-speed gearbox functions in both directions of rotation; an adaptation of the pockets or recesses for the blocking elements 42 within the ring gear carrier 30 may possibly be necessary in order to permit an adequate clamping action. Instead of a spring 46, control pins 44 arranged on both sides can also be assigned to the respective blocking element 42 in order to permit a guided movement into a released position or an engaged position.

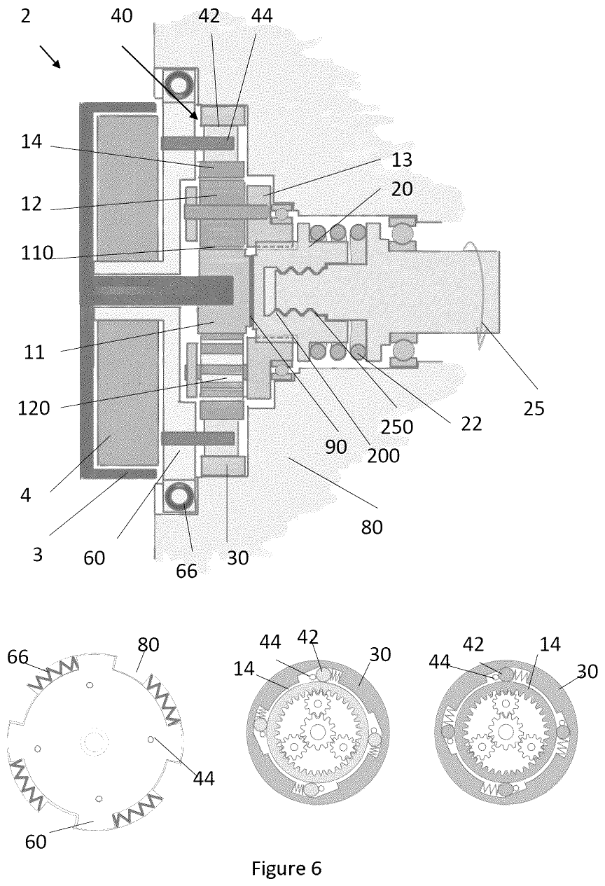

FIG. 6 illustrates a variant of the change-speed gearbox in which the same components have the same designations. The basic structure of the change-speed gearbox is unchanged; here, too, the ring gear 14 is mounted within the ring gear carrier 30 via a switchable freewheel 40. One difference results from the configuration of the output element 20, which is coupled to the output component 25 via a threaded connection. Formed within the bore in the output element 20 is an internal thread 200, which engages in an external thread 250 on the end of the output component 25 that faces the planetary gearbox 10. Instead of an actuator which displaces the control pins 44, the control pins 44 in the embodiment according to FIG. 6 are fastened to a stator carrier 60 which is supported so as to be movable, in particular rotatable, within the stationary component 80. A plurality of springs 66 are assigned protrusions on the stator carrier 60 and are supported on corresponding protrusions in a respective recess within the stationary component 80. This is illustrated in the lower left-hand illustration of FIG. 6. A total of four springs 66 are arranged to be distributed around the circumference on the stator carrier 60 and are supported on the stationary component 80, for example an upper part of a joint device.

If only a low torque is needed to adjust the output component 25, the spring 22 urges the output element 20 in the direction of an engaged controllable clutch 90 via the threads 200, 250, and the springs 66 hold the control pins 44 in the released position, which is shown in the center illustration of the lower row of FIG. 6. The ring gear 14 can rotate freely within the ring gear carrier 30, the clutch 90 is engaged and the drive 2, with a high rotational speed and direct transmission, rotates the output element 20 and the output component 25 at the motor rotational speed.

If a higher resistance torque is opposed to the fast adjustment provided with a low torque, for example if a prosthetic gripping device detects an object, the springs 66 which support the stator carrier 60 compress and pivot the control pins 44 counter-clockwise. The blocking elements 42 are then clamped with the ring gear carrier 30, which is shown in the lower right hand illustration, so that the ring gear 14 is blocked. The effect of the higher resistance torque is that a relative movement takes place between the output component 25 and the output element 20, so that the threads 200, 250 lead to the clutch 90 being disengaged. The force is then transmitted from the rotor 3 to the output component 25 via the sun gear 11 and the planet gears 12, via the planet carrier 13 and the inner toothing formed therein and the external toothings on the output element 20,

One advantage of this embodiment consists in the fact that no switching motor or actuator is needed in order to make the changeover from a high gear at a high rotational speed and lower torque transmission to a low gear with a lower rotational speed and a high torque. The changeover is made automatically under torque control and as a function of the load and also purely mechanically, so that no separate actuator, no sensors and no electronic control have to be used.

The change-speed gearbox in both embodiments needs no linearly acting transmission of force from the motor to the output component and is extraordinarily flexible with regard to the selection of the motor. The gearbox can be arranged very flexibly within the transmission path; in particular the change-speed gearbox can be arranged close to the motor or drive, as a result of which long transmission paths, associated losses and increased assembly play are dispensed with. Alternatively, the change-speed gearbox can be arranged close to the output, as a result of which drive elements upstream of the change-speed gearbox are protected against higher loads. The switching travels are very short, so that a changeover between the individual gears can be made quickly. As a result of the use of a planetary gearbox with a planet stage, a compact and lightweight design can be achieved. It is possible to carry out a changeover from a high gear to a low gear under load. For the changeover with an actuator, only a single motor is needed, a directional changeover or a reversal of the direction of rotation of the change-speed gearbox is not needed for a changeover between different gears. Because of the unidirectional nature of the change-speed gearbox, gear changing is carried out only in one direction, wherein the gearbox automatically resets to a standard gear in the other direction. This is very advantageous in many joints of the lower extremities and upper extremities since a higher gear is often preferred in one direction, and therefore no active control of the change-speed gearbox is needed in order to switch from the low, slow gear to the high, fast gear. Furthermore, automated, load-dependent switching without sensor control is possible via the mechanical solution.

### LIST OF DESIGNATIONS

2 Drive

3 Rotor

4 Stator

10 Planetary gearbox

11 Sun gear

12 Planet gear

13 Planet carrier

14 Ring gear

20 Output element

22 Compression spring

25 Output component

30 Ring gear carrier

40 Freewheel

42 Blocking element

44 Control pin

46 Spring

50 Control sleeve

60 Stator carrier

66 Spring

70 Actuator

75 Gear wheel

80 Stationary component

90 Clutch

110 External toothing

120 External toothing

140 Internal toothing

200 Internal thread

250 External thread

350 Carrier thread

500 Toothing

530 Adjustment thread

750 Toothing

1. Change-speed gearbox for orthopedic devices, comprising:
a planetary gearbox comprising
at least one planet stage having a central sun gear,
a plurality of planet gears rotatably mounted on a planet carrier, wherein the plurality of planet gears come into engagement with the central sun gear, and
a ring gear which meshes with the plurality of planet gears;
a drive coupled to or couplable to the central sun gear; and
an output element coupled to the planet carrier,
wherein the ring gear is pivotably mounted in a ring gear carrier and is coupled to the ring gear carrier via a switchable freewheel.

2. The change-speed gearbox according to claim 1, wherein the switchable freewheel is torque-controlled or is designed such that it is changeable via an actuator.

3. The change-speed gearbox according to claim 1 further comprising a controllable clutch arranged between the central sun gear and the output element.

4. The change-speed gearbox according to claim 1 wherein the output element is axially displaceably mounted on the planet carrier.

5. The change-speed gearbox according to claim 4, wherein the output element is spring-biased in a direction of the central sun gear.

6. The change-speed gearbox according to claim 5, wherein the output element is mounted on a thread, or wherein the output element is torsionally rigidly and axially displaceably mounted on an output component.

7. The change-speed gearbox according to claim 1 wherein the switchable freewheel comprises at least one blocking element which is spring-loaded by a spring having a spring force in a direction of an engaged position, wherein the at least one blocking element is in force-transmitting engagement with the ring gear and the ring gear carrier, and wherein the blocking element is assigned at least one control pin which moves the blocking element counter to the spring force into a released position in which the blocking element is out of engagement.

8. The change-speed gearbox according to claim 1 wherein the output element is coupled to a control sleeve that is rotatable relative to the ring gear carrier.

9. The change-speed gearbox according to claim 7 wherein the at least one control pin is mounted on a control sleeve that is rotatable relative to the ring gear carrier, or wherein the at least one control pin is mounted on a stator carrier that is rotatable relative to the ring gear carrier.

10. The change-speed gearbox according to claim 8 wherein the control sleeve is coupled to the actuator.

11. The change-speed gearbox according to claim 8 wherein the control sleeve is coupled to the actuator by a frictional fit or via toothing.

12. The change-speed gearbox according to claim 8 wherein the control sleeve comprises an adjustment thread which meshes with a carrier thread.

13. The change-speed gearbox according to claim 12, wherein the carrier thread is arranged on or formed on the ring gear carrier, or the carrier thread is arranged on or formed on a component coupled in a fixed manner to the ring gear carrier.

14. The change-speed gearbox according to claim 8 wherein the control sleeve is formed such that in a first position of the control sleeve the clutch is engaged and the switchable freewheel is kept out of engagement.

15. The change-speed gearbox according to claim 8 wherein the control sleeve is formed such that in a second position of the control sleeve the clutch is disengaged and the switchable freewheel is kept out of engagement.

16. The change-speed gearbox according to claim 8 wherein the control sleeve is formed such that in a third position of the control sleeve the clutch is disengaged and the switchable freewheel is kept in engagement.

17. The change-speed gearbox according to claim 9, wherein the stator carrier is mounted so as to be spring-biased in a direction of an initial position in which the switchable freewheel is kept out of engagement.

18. The change-speed gearbox according to claim 17, wherein the stator carrier is mounted on the ring gear carrier, or wherein the stator carrier is mounted on a component coupled in a fixed manner to the ring gear carrier.

19. The change-speed gearbox according to claim 1 wherein the planet stage is designed as a toothed gear stage, wherein the central sun gear has external toothing, wherein each of the plurality of planet gears are provided with external toothing systems which mesh with the external toothing of the central sun gear, and wherein the ring gear has internal toothing which meshes with the external toothing of the plurality of planet gears.

20. The change-speed gearbox according to claim 1, wherein the change-speed gearbox is configured to be changeable between two transmission ratios in a first direction of rotation, and, in an opposite second direction of rotation, is switched to a non-switchable standard transmission ratio.
